(19) United States
(12) Patent Application Publication
(10) Pub. No.: US 2011/0196318 A1
(43) Pub. Date: Aug. 11, 2011

(54) GEL-BASED PHENERGAN COMPOSITION AND METHOD OF DELIVERY

(76) Inventor: Delmer H. Parrish, Naples, FL (US)

(21) Appl. No.: 12/702,814

(22) Filed: Feb. 9, 2010

(51) Int. Cl. A61M 5/315; A61K 31/5415; A61P 1/08; A61M 5/178

(52) U.S. Cl. 604/290; 514/226.2; 604/310; 604/311

(57) ABSTRACT

The invention is directed to a system for treating vomiting comprising a topical gel placed within a syringe. The topical gel includes (a) between 10 to 40% Phenergen by weight, (b) between 30 to 50% of lecithin isopropyl palmitate solution by weight, and (c) between 30 to 70% of pluronic gel by weight. The syringe includes a cylindrical chamber having a front end, a back end, an interior side and exterior side. The syringe has a disposing opening located on the front end of the cylindrical chamber; a stopper of a sufficient size and dimension to engage the disposing opening; a plunger located within the cylindrical chamber; a rigid shaft having a sufficient size and dimension to fit within the cylindrical chamber and a plate affixed to a distal end of the rigid shaft. The syringe is plastic having a dark color to prevent degradation of the topical gel.

### FIELD OF THE INVENTION

This invention is generally directed to a gel-based carrier sufficient to maintain promethazine based medical compositions (known as “Phenergan”) for placement on a patient's skin. More specifically, the technology is directed to a method to reduce vomiting (and resulting dehydration) by applying a topical amount of gel containing Phenergan onto a patient's skin.

### BACKGROUND OF THE INVENTION

Promethazine is a first-generation H₁ receptor antagonist used medically as both an antihistamine and antiemetic. Its chemical formula is C₁₇H₂₀N₂S and has a molecular mass of 284.42 grams/mol. Promethazine compositions are given by prescription in the United States and sold over-the-counter in most European countries. In the United States, promethazine is typically and commonly referred to as “Phenergan.”

Phenergan, when in the form promethazine hydrochloride, appears as a white to faint yellow crystalline powder that is essentially odorless. When prepared as a hydrochloride salt, the composition is water soluble and somewhat soluble in alcohol. There is a risk however of slow oxidation after prolonged exposure to air which results in a blue discoloration.

Phenergan has a variety of medical effects upon administration to a patient. Often, Phenergan is prescribed to treat symptoms such as itching, sneezing, water eyes or common skin rashes. However, Phenergan can also be employed to control vomiting due to a variety of conditions such as flu, food poising, food allergies, upset stomach, chemotherapy, and the common cold. Only minor side effects have been reported in a small subset of individuals treated with Phenergan, including dry mouth, dizziness, fatigue, dry mouth and constipation.

Currently, there exist three primary administration techniques for using Phenergan to treat vomiting—all of which have significant drawbacks. First, Phenergan can be taken directly by the patient in tablet (pill) form. However, it is often impossible to take Phenergan orally because the patient may be vomiting such that the pill will never be absorbed in the digestive system.

A second method of treatment is through use of a suppository. This typically requires administration by a third-party, by way of example a family member, while holding the patient down on a bed or on the floor. This form is loathed for obvious reasons, including great discomfort to the patient. Suppository administration is often traumatic for young children suffering from vomiting.

A third currently used system for administering Phenergan is through use of a syringe which invariably requires assistance by a medical professional. Under this third system, the patient must travel to a medical facility (like a doctor's office, clinic, out-patient facility or even a hospital) to receive an injection of Phenergan. This can be highly expensive as well as uncomfortable, as the patient may not be in a condition contusive to travel. As an alternative, a home health nurse or nurse's aide can be dispatched to the home for syringe administration—but this is still an expensive process and risks delay of medical treatment.

All three examples provided above constitute the current available methods for delivery of Phenergan to help curve vomiting resulting for certain medical conditions. Without quick and efficient treatment of vomiting, patients may risk dehydration and other resulting medical conditions, which may require long-term hospitalization. This is especially true with children and the elderly. Accordingly, there is a need in treatment of vomiting for a more effective, low cost and reliable medical therapy. In addition, there is a need for a robust method of treatment of vomiting that does not require administration of the medicine via tablet or suppository.

### SUMMARY OF THE INVENTION

The present invention solves many of the current limitations presently found in treating the medical condition of vomiting often associated with the flu, food allergy, food poisoning, chemotherapy, and the common cold. More specifically, the invention relates to a water based topical gel, which acts as a carrier to a prescribed quantity of Phenergan. A pharmacist (or other qualified medical professional) can add the correct amount of Phenergan necessary to treat a given patient based upon their age, weight and the underlying medical condition (as well as other pre-existing conditions).

The invention is first directed toward a system for treating the medical condition comprising a topical gel placed within a syringe. The topical gel may include: (a) a quantity of Phenergan, where the Phenergan is between 20 to 40 percent, by weight, of the topical gel; (b) a quantity of lecithin isopropyl palmitate solution, where the lichitin isopropyl palmitate solution is between 30 and 50, by weight, of the topical gel; and (c) a quantity of pluronic gel, which is between 30 and 70 percent, by weight of the topical gel.

The syringe may include a cylindrical chamber having a front end, a corresponding back end, an interior side and a corresponding exterior side. In addition, the syringe has a disposing opening located on the front end of the cylindrical chamber and a stopper of a sufficient size and dimension so as to engage and close the disposing opening. Also located within the cylindrical chamber is a plunger. A rigid shaft is affixed to plunger. The rigid shaft has a sufficient size and dimension to fit within the interior side of the cylindrical chamber. Also, the rigid shaft extends outside of the back end of the cylindrical chamber. A plate can be affixed to the distal end of the rigid shaft to assist disbursement of the topical gel. The syringe is preferably made of a plastic, which has a dark color to prevent degradation of the topical gel. In addition, the exterior side of the cylindrical chamber should include a volumetric ruler to aid in disbursing the correct amount of topical gel.

The invention is further directed toward a method of treating the medical condition of vomiting. The method may first includes the step of preparing the aforementioned topical gel to include Phenergan, lecithin isopropyl palmitate solution, and a quantity of pluronic gel. Next, the method calls for placing the topical gel into a syringe and then disbursing a predetermined quantity of topical gel onto the skin of a patient. Next, the topical gel is absorbed onto the skin of the patient—which can include rubbing the topical gel. The surfactant qualities of the topical gel assists absorption into the skin. Each disbursement of topical gel can be between 1.0 to 3.0 ml for an adult patient. These four steps can be repeated every four to six hours as needed until vomiting subsides.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1 is a diagram showing the preferred ingredients of the topical gel; and

FIG. 2 is a front view of a syringe for administrating topical gel containing Phenergan onto a patient.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete and will fully convey the scope of the invention to those skilled in the art. Like numbers refer to like elements throughout.

### Preparation of the Topical Gel

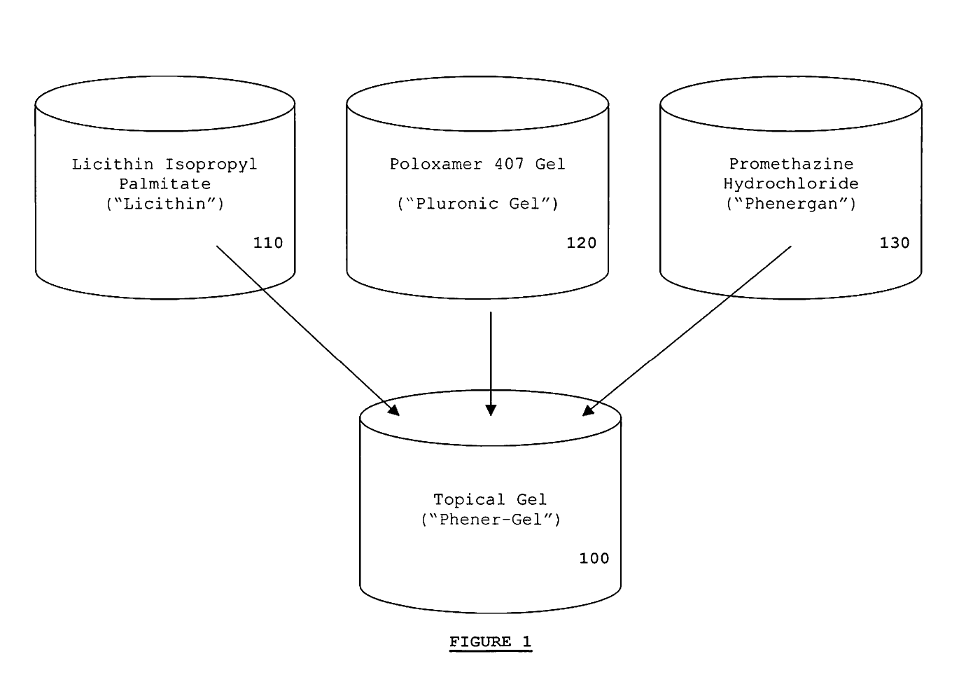

As illustrated in FIG. 1, the topical gel 100 (one embodiment is herein presented by way of example) is prepared through combination of three components: lecithin isopropyl palmitate solution 110 (referred to as “lecithin”), poloxamer 407 gel 120 (referred to as “pluronic gel”), and Phenergan 130. Under this composition of the topical gel 100, both the lichithin 110 and pluronic gel 120 act as a base.

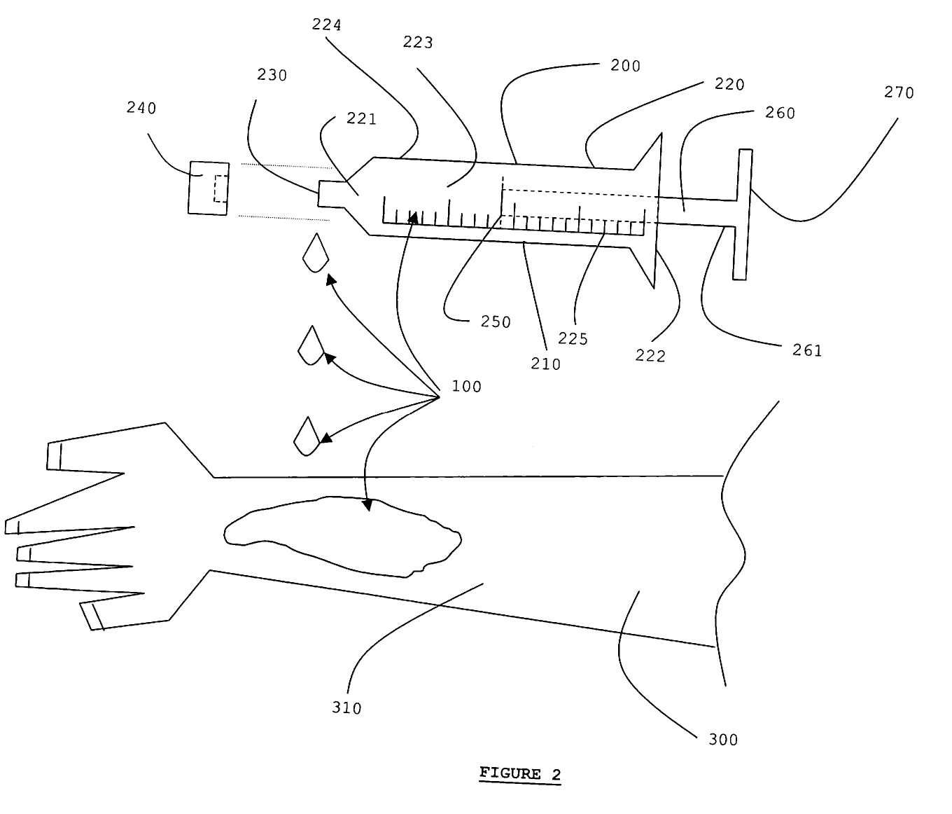

Use of licethin 110 as one component of the topical gel 100 has multiple benefits. Licethin is a viscous tan solution that acts as a surfactant as well as a delivery device for other pharmaceutical agents. As a surfactant, licethin 110 can help not only carry Phenergan 130, but also help its administration onto the skin 310 of the patient 300 (both shown in FIG. 2). Other surfactants with similar chemical compositions and properties of licethin 110, known to those of ordinary skill in the art, can also be used to form the topical gel 100.

Polaxamer 407, also referred to as its commercial name pluronic gel 120, is a hydrophilic and non-ionic surfactant of the more general class of copolymers known as poloxamers. Pluronic gel 120 is a triblock copolymer consisting of a central hydrophobic block of polyproplyene glycol flanked by two hydrophilic blocks of polyethylene glycol. The approximate lengths of the two PEG blocks is 101 repeat units while the approximate length of the propylene glycol block is 56 repeat units. Uses of pluronic gel 120 include cosmetics, contact lens cleaning solutions and some mouthwashes. The pluronic gel 120 component can be commercially available Plutonic F127 Gel. Other copolymers similar in chemical composition and properties of pluronic gel 120, know to those of ordinary skill in the art, can also be used as a base for the topical gel 100.

As further illustrated in FIG. 1, the topical gel 100 is created by first incorporating a quantity of 1.0 to 2.0 Gm of Phenergan 130 into 16 to 20 ml of pluronic gel 120. The Phenergen 130 component can be commercially available promethazine hydrochloric USP. Next, between 6 to 7 ml of lecithin 110 is added. As the third step, the volume of the compound is brought to 30 mL with an addition of Phenergel 130. During performance of these steps, it is preferable to use a dremel tool with a mixing blade or ointment mill to add the mixing of these various components 110-130. Once created, the topical gel 100 should be maintained in a low lighted area and/or in a container that prevents direct access to natural or artificial light.

Put another way, the composition of topical gel 100 preferably includes:

| | | |
|---|---|---|
| Promethazine Hydrochloric USP | 1.5 Gm | 13% weight |
| Lecithin Isopropyl Palmitate Solution | 6.6 ml | 22% weight |
| Poloxamer 407 Gel 20% | 23.0 ml | 65% weight |

Preferably, Phenergen 130 is between 10 to 40 percent, by weight, of the topical gel 100. Likewise, licithin 110 is preferably 30 to 50 percent, by weight, of the topical gel 100. Finally, pluronic gel 120 is between 30 and 70 percent, by weight, of the topical gel 100.

### Treatment Device

In addition to a composition of topical gel 100, the invention is further directed toward a treatment device to prevent vomiting caused by a variety of medical conditions. FIG. 2 illustrates one form of instrument 200 for administering the topical gel 100. As shown, the instrument 200 can be a syringe 210.

By way of example for one embodiment, the syringe 210 includes a cylindrical chamber 220, a disposing opening 230, and a stopper 240 of a sufficient size and dimension to engage and close the disposing opening 230. The cylindrical chamber 220 includes a front end 221, a corresponding back end 222, and interior side 223 and a corresponding exterior side 224. Positioned within the interior side 223 of the cylindrical chamber 220 is a plunger 250 attached to a rigid shaft 260. The plunger 250 is capable of maintaining topical gel 100 within the interior side 223 of the cylindrical chamber 220 and the disposing opening 230. The syringe 210 is preferably made of a biodegradable and hypoallergenic plastic or any similar material, know to those of ordinary skill in the art. Moreover, the syringe 210 is preferably made of a dark amber material to prevent light from degrading the stored topical gel 100.

FIG. 2 further illustrates the syringe 210 including a plate 270 attached at one distal end 261 of the rigid shaft 260. The shaft 260 is of sufficient side and dimension to fit and seal within the interior side 221 of the cylindrical chamber 220. Through pressing the plate 270 to force the shaft 260 within the cylindrical chamber 220, the plunger 250 in turn makes contact with the topical gel 110. This pressure allows a prescribed amount of topical gel 110 to be squeezed and released through the disposing opening 230.

As further shown in FIG. 2, the exterior side 222 of the cylindrical chamber 220 includes a volumetric ruler 225. This volumetric ruler 225 allows measurement of a finite amount of topical gel 100 to be administered through the syringe 210 onto a patient 300. Preferably, the volumetric ruler 225 can show specific measures for up to 3.0 ml of topical gel 100.

It is preferable, but not necessary, for an administrator (not shown) to administer the topical gel 100 onto the patient 300 through use of the syringe 210. The administrator can be a spouse, family member, acquaintance or a medical professional. When providing this service, it is recommended that the administrator wear some sort of covering to prevent direct contact with the topical gel 100—in order to lessen the risk of potential side effects. Such covering (also not shown) can include latex gloves, a latex finger sleeve or similar protection.

### The Method of Treatment

The invention is further directed to a method of treating the medical condition of vomiting. For one embodiment herein presented by way of example, a first step of the method is to prepare the topical gel 100. As shown in FIG. 1, the topical gel 100 has at least three components, which first includes a quantity of Phenergan 130, preferably a commercially available quantity of promethazine hydrochloric USP, which is between 10 to 40 percent by weight of the topical gel 100. The second ingredient is a quantity of lecithin 110, where the lecithin 110 is between 30 and 50 percent by weight of the topical gel 100. The third ingredient is a quantity of pluronic gel 120, which is between 30 and 70 percent, by weight of the topical gel 100.

After creating the topical gel 100, the method next contemplates placing this prepared topical gel 100 into a syringe 210. As shown in FIG. 2, the syringe 210 includes a cylindrical chamber 220 that includes a front end 221, a corresponding back end 222, an interior side 223 and a corresponding exterior side 224. In addition, the syringe 210 includes a disposing opening 230 located on the front end 221 of the cylindrical chamber 220.

As further shown in FIG. 2, a stopper 240 of a sufficient size and dimension so as to engage and close the disposing opening 230 is included. Located within the interior side 223 of the cylindrical chamber 220 is a plunger 250. A rigid shaft 260 is affixed to the plunger 250. The rigid shaft 260 has a sufficient size and dimension to fit within the interior side 223 of the cylindrical chamber 200. A plate 270 affixed to a distal end 261 of the rigid shaft 260 helps squeeze the topical gel 100 out of the syringe 210.

The next step of the method is disbursing a predetermined quantity of topical gel 100 onto the patient 300. An adult patient 300 should be given a dose between 1.0 ml and 3.0 ml of topical gel 100 per treatment. Likewise, children should be given a dose of 0.12 ml per dose, while infants should be given half of the children's dose. Once disbursed, the topical gel 100 should be absorbed onto the patient 300. This can include the gentle rubbing of the topical gel on an area of skin. These steps can be repeated ever four to six hours as needed until the vomiting subsides.

I claim:

1. A topical gel for treatment of the medical condition of vomiting, the topical gel comprising:
(a) a first quantity of Phenergan, wherein the Phenergan is between 10 to 40 percent, by weight, of the topical gel;
(b) a second quantity of lecithin isopropyl palmitate solution, wherein the lichitin isopropyl palmitate solution is between 30 and 50, by weight, of the topical gel; and
(c) a third quantity of pluronic gel, wherein the third quantity of pluronic gel is between 30 and 70 percent, by weight of the topical gel.

2. The topical gel of claim 1, wherein:
(a) the amount of Phenergan is generally 13 percent of the topical gel;
(b) the amount of lecithin isopropyl palmitate solution is generally 22 percent of the topical gel; and
(c) the amount of pluronic gel is generally 65 percent of the topical gel.

3. The topical gel of claim 1, wherein:
the pluronic gel component is commercially available Plutonic F127 Gel.

4. The topical gel of claim 1, wherein:
the Phenergen component is commercially available promethazine hydrochloric USP.

5. A system for the treatment of the medical condition of vomiting, the system comprising:
a topical gel placed within a syringe, the topical gel including:
(a) a quantity of Phenergan, wherein the Phenergan is between 10 to 40 percent, by weight, of the topical gel;
(b) a quantity of lecithin isopropyl palmitate solution, wherein the lichitin isopropyl palmitate solution is between 30 and 50, by weight, of the topical gel; and
(c) a quantity of pluronic gel, wherein the quantity of Phenergan is between 30 and 70 percent, by weight of the topical gel.

6. The system of claim 5, wherein the syringe includes:
a cylindrical chamber having a front end, a corresponding back end, an interior side and a corresponding exterior side;
a disposing opening located on the front end of the cylindrical chamber;
a stopper of a sufficient size and dimension so as to engage and close the disposing opening;
a plunger located within the interior side of the cylindrical chamber;
a rigid shaft affixed to the plunger, the rigid shaft having a sufficient size and dimension to fit within the interior side of the cylindrical chamber, the rigid shaft extending outside of the back end of the cylindrical chamber; and
a plate affixed to a distal end of the rigid shaft.

7. The system of claim 6, wherein the syringe is made of a plastic.

8. The system of claim 6, wherein the syringe is made of a dark color sufficient to prevent degradation of the topical gel.

9. The system of claim 5, wherein:
(a) the amount of Phenergan is generally 13 percent of the topical gel;
(b) the amount of lecithin isopropyl palmitate solution is generally 22 percent of the topical gel; and
(c) the amount of pluronic gel is generally 65 percent of the topical gel.

10. The system of claim 5, wherein:
the pluronic gel component is commercially available Plutonic F127 Gel.

11. The system of claim 5, wherein:
the Phenergen component is commercially available promethazine hydrochloric USP.

12. A method of treating the medical condition of vomiting, the method comprising the steps of:
(a) preparing a topical gel, the topical gel having at least three components which include:
(i) quantity of Phenergan, wherein the Phenergan is between 20 to 40 percent, by weight, of the topical gel
(ii) a quantity of lecithin isopropyl palmitate solution, wherein the lichitin isopropyl palmitate solution is between 30 and 50, by weight, of the topical gel, and
(iii) a quantity of pluronic gel, wherein the quantity of Phenergan is between 30 and 70 percent, by weight of the topical gel;
(b) placing the topical gel into a syringe;
(c) disbursing a predetermined quantity of topical gel onto skin of a patient; and
(d) absorbing the disbursed topical gel by the skin of the patient.

13. The method of claim 12, further comprising the additional step of:
(e) repeating steps (a)-(d) ever four to six hours as needed until the vomiting subsides.

14. The method of claim 12, wherein the syringe includes:
a cylindrical chamber having a front end, a corresponding back end, an interior side and a corresponding exterior side;
a disposing opening located on the front end of the cylindrical chamber;
a stopper of a sufficient size and dimension so as to engage and close the disposing opening;
a plunger located within the interior side of the cylindrical chamber;
a rigid shaft affixed to the plunger, the rigid shaft having a sufficient size and dimension to fit within the interior side of the cylindrical chamber, the rigid shaft extending outside of the back end of the cylindrical chamber; and
a plate affixed to a distal end of the rigid shaft.

15. The method of claim 14, wherein the syringe is made of a plastic.

16. The method of claim 14, wherein the syringe is made of a dark color sufficient to prevent degradation of the topical gel.

17. The method of claim 12, wherein:
(a) the amount of Phenergan is generally 13 percent of the topical gel;
(b) the amount of lecithin isopropyl palmitate solution is generally 22 percent of the topical gel; and
(c) the amount of pluronic gel is generally 65 percent of the topical gel.

18. The method of claim 12, wherein the pluronic gel component is commercially available Plutonic F127 Gel.

19. The method of claim 12, wherein the Phenergen component is commercially available promethazine hydrochloric USP.

20. The method of claim 12, wherein each dose of topical gel should be between 1.0 to 3.0 ml for an adult patient.
